(19) United States
(12) Patent Application Publication
(10) Pub. No.: US 2010/0036119 A1
(43) Pub. Date: Feb. 11, 2010

(54) FLUORINATING REAGENTS AND THEIR PREPARATION

(75) Inventors: WOLFGANG EBENBECK, LEVERKUSEN (DE); PETRA HILGERS, ROSRATH (DE); ALBRECHT MARHOLD, LEVERKUSEN (DE); JAN ALEXANDER BARTEN, BREMEN (DE); ALEXANDER KOLOMEITSEV, BREMEN (DE); GERD-VOLKER ROSCHENTHALER, BREMEN (DE)

Correspondence Address: LANXESS CORPORATION, 111 RIDC PARK WEST DRIVE, PITTSBURGH, PA, 15275-1112, US

(73) Assignee: LANXESS DEUTSCHLAND GMBH, LEVERKUSEN (DE)

(21) Appl. No.: 12/580,821

(22) Filed: Oct. 16, 2009

Related U.S. Application Data
(62) Division of application No. 10/751,623, filed on Jan. 5, 2004.

(30) Foreign Application Priority Data
Jan. 7, 2003 (DE) 10300112.3

(51) Int. Cl. C07C 209/50; C07C 211/03; C07D 265/30; C07D 213/38; C07D 207/32; C09K 3/00

(52) U.S. Cl. 544/162; 564/510; 546/329; 548/566; 252/183.11

(57) ABSTRACT

The present invention relates to α,α-difluoroamines, fluorinating reagents comprising α,α-difluoroamines and also processes for preparing α,α-difluoroamines and fluorinating reagents comprising α,α-difluoroamines.

This application is a divisional of U.S. patent application Ser. No. 10/751,623 filed Jan. 5, 2004, entitled “Fluorinating Reagents and Their Preparation”, the contents of which are hereby incorporated by reference in their entirety.

### BACKGROUND OF THE INVENTION

1. Field of the Invention

The present invention relates to α,α-difluoroamines, fluorinating reagents comprising α,α-difluoroamines and processes for preparing an using them.

2. Brief Description of the Prior Art

Known flourinating reagents for, say, fluorinating alcohols or carbonyl compounds, in particular ketones, carboxylic acids and aldehydes, are, for example, sulphur tetrafluoride, diethylaminosulphur trifluoride (DAST) and bis(methoxyethyl)aminosulphur trifluoride (methoxy-DAST) (see U.S. Pat. No. 3,976,691, EP-A 90 448 and EP-A 905 109).

A disadvantage of the industrial use of sulphur tetrafluoride is its extremely high toxicity and the necessity of extensive safety measures. The diethylaminosulphur trifluorides are additionally shock-sensitive (J. Fluorine Chem. 1989, 42, 137) and, as a consequence of their explosiveness, are subject to strict legal provisions.

A further reagent for fluorinating secondary alcohols and carboxylic acids is N,N-dimethyl-1,1-difluorobenzylamine which is obtainable by reacting N,N-dimethylbenzamide with sulphur tetrafluoride at 150° C. [J. Fluorine Chem. 1983, 23, 219-228]. However, the breadth of application of the reagent is restricted and it affords only moderate yields.

Another known fluorinating reagent for alcohols is 2-chloro-1,1,2-trifluorotriethylamine, known as the Yarovenko reagent (Org. React. 1974, 21, 158). However, the reagent is not storage-stable and can only be prepared with great difficulty.

Yet another reagent known as Ishikawa reagent consists of a mixture of hexa-fluoropropyldialkylamine and pentafluoroalkenyl-dial-kylamine. However, this reagent has the same abovementioned disadvantages.

EP-A 895 991 discloses difluoromethylene-α,α-diazo compounds which can be used for fluorinating hydroxyl and carboxyl functions. As a consequence of their high sensitivity to air and moisture, they are only of limited suitability for industrial use.

There is, therefore, the need to provide fluorinating reagents which can be prepared efficiently from readily available reactants, are storage-stable and can fluorinate the hydroxyl and ketone functions in good yields.

### SUMMARY OF THE INVENTION

Compounds of the formula (I) have now been found

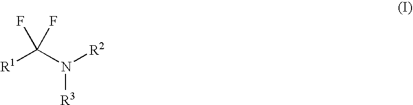

in which
R¹ is hydrogen, C₁-C₁₂-alkyl, [(C₂-C₁₂-alkylene)-O]ₙ(C₁-C₁₂-alkyl)] where n=1 to 5, C₄-C₁₅-arylalkyl or C₃-C₁₄-heteroaryl,
R² and R³ are each independently C₄-C₁₅-arylalkyl or C₁-C₁₂-alkyl, or together are part of a cyclic radical having a total of 3 to 12 carbon atoms or
R¹ and R² and/or R³ together are part of a cyclic radical having a total of 3 to 12 carbon atoms,
   excluding 1,1-difluormethyl-N,N-dimethylamine, 1,1-difluormethyl-N,N-diethylamine, 1,1-difluormethyl-N,N-diisopropylamine and 1,1-difluoro-N,N-2-trimethyl-1-propanamine.

In the context of the invention, all radical definitions and parameters given, either in general or within areas of preference, i.e. the particular areas and areas of preference too, may be combined with each other as desired.

It should be noted that the illustration of the formula (I) which has been selected for reasons of simplification also encompasses the illustration below which is often used in the literature.

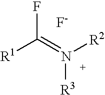

The same applies similarly in the context of the invention for all illustrations and nomenclatures of α,α-dihaloamine functionalities.

### DETAILED DESCRIPTION OF THE INVENTION

Alkyl, alkylene and alkoxy are in each case independently a straight-chain, cyclic, branched or unbranched alkyl, alkylene and alkoxy radical respectively. The same applies to the aromatic moiety of an arylalkyl radical.

C₁-C₄-alkyl is, for example, methyl, ethyl, n-propyl, isopropyl, n-butyl, sec-butyl and tert-butyl, C₁-C₈-alkyl is additionally, for example, n-pentyl, 1-methylbutyl, 2-methylbutyl, 3-methylbutyl, neopentyl, 1-ethylpropyl, cyclohexyl, cyclopentyl, n-hexyl, 1,1-dimethyl-propyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl and n-octyl, and C₁-C₁₂-alkyl is still further additionally, for example, adamantyl, the isomeric menthyls, n-nonyl, n-decyl and n-dodecyl.

C₁-C₄-alkoxy is, for example, methoxy, ethoxy, n-propoxy, isopropoxy, n-butoxy, sec-butoxy and tert-butoxy, C₁-C₈-alkoxy is additionally n-pentoxy, 1-methylbutoxy, 2-methylbutoxy, 3-methylbutoxy, neopentoxy, 1-ethylpropoxy, cyclohexoxy, cyclopentoxy, n-hexoxy and n-octoxy, and C₁-C₁₂-alkoxy is still further additionally, for example, adamantoxy, the isomeric menthoxy radicals, n-decoxy and n-dodecoxy.

C₂-C₁₂-alkylene is, for example, 1,2-ethylene, 1,3-propylene, 1,4-butylene, 1,2-cyclo-hexoxylene and 1,2-cyclopentylene.

Heteroaryl is in each case independently a heteroaromatic radical having 3 to 14 frame-work carbon atoms of which no, one, two or three framework carbon atoms per cycle, but at least one framework atom in the entire molecule, is also selected from the group of nitrogen, sulphur and oxygen.

Examples of heteroaromatic radicals are pyridinyl, oxazolyl, benzofuranyl, dibenzofuranyl and quinolinyl.

The heteroaromatic radical may also be substituted by up to five identical or different substituents per cycle which are selected from the group of chlorine, fluorine, C₁-C₁₂-alkyl, C₁-C₁₂-fluoroalkyl, C₁-C₁₂-fluoroalkoxy, C₁-C₁₂-fluoroalkylthio, C₁-C₁₂-alkoxy, di(C₁-C₈-alkyl)amino and tri(C₁-C₈-alkyl)siloxyl.

Aryl is in each case independently a heteroaryl radical as defined above or a carbocyclic aromatic radical.

Examples of carbocyclic aromatic radicals having 6 to 14 framework carbon atoms are phenyl, naphthyl, phenanthrenyl, anthracenyl and fluoronyl.

The carbocyclic aromatic radical may also be substituted as described above for the heteroaromatic radicals.

Arylalkyl is in each case independently a straight-chain, cyclic, branched or unbranched alkyl radical as defined above which may be singly, multiply or fully substituted by aryl radicals as defined above.

The preferred substitution patterns for compounds of the formula (I) are defined herein-below:
R¹ is preferably hydrogen, C₁-C₁₂-alkyl, or C₃-C₅-heteroaryl, more preferably hydrogen or C₁-C₈-alkyl and most preferably hydrogen or C₁-C₄-alkyl.
R² and R³ are preferably each independently C₁-C₈-alkyl or NR²R³ as a whole is N-morpholinyl, N-methyl-1,4-piperazin-N-yl, and more preferably each identically methyl, ethyl or isopropyl.

The compounds of the formula (I) include:
1,1-difluoro-N,N-2,2-tetramethyl-1-propanamine, N,N-diethyl-α,α-difluoro-2,2-dimethyl-1-propanamine, N-(1,1-difluoromethyl)morpholine, N,N-diethyl-α,α-difluoro-3-pyridylmethanamine, N,N-diethyl-α,α-difluoro-2-pyridylmethanamine and 2,2-difluoro-1,3,3-trimethylpyrrolidine.

Preference is given to the compounds of formula (I) as a whole being 2,2-difluoropyrrolidin, 2,2-difluoropiperidine, [2.2.2]-2,2,5,5-tetrafluoro-1,4-diazabicyclooctane or [2.2.2]-2,2,6,6-tetrafluoro-1,4-diazabicyclooctane, and the radicals mentioned may optionally be mono- or polysubstituted by C₁-C₄-alkyl.

It has been found that, surprisingly, the compounds of the formula (I) according to the invention, function more efficiently as fluorinating reagents when they are used in the presence of a tertiary aprotic amine and/or of an N-heteroaromatic compound and in the present of hydrogen fluoride.

The invention therefore also encompasses mixtures comprising
Compounds of the formula (Ia)

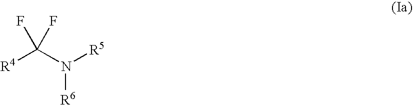

in which
R⁴ is hydrogen, C₁-C₁₂-alkyl, [(C₂-C₁₂-alkylene)-O]ₙ(C₁-C₁₂-alkyl)] where n=1 to 5, C₃-C₁₄-aryl or NR⁷R⁸ where R⁷ and R⁸ are each independently C₁-C₈-alkyl, or NR⁷R⁸ as a whole is a 4- to 7-membered cyclic radical having a total of 3 to 12 carbon atoms and
R⁵ and R⁶ are each independently C₁-C₁₂-alkyl or are together part of a cyclic radical having a total of 4 to 12 carbon atoms or
R⁴ and R⁵ and/or R⁶ together are part of a cyclic radical having a total of 4 to 12 carbon atoms,
   at least one aprotic, tertiary amine which contains no fluorine atoms in the α-position to the nitrogen and/or at least one N-heteroaromatic compound and
   hydrogen fluoride.

In this context, aprotic means that the tertiary amine which may also be a molecule having a plurality of tertiary amino groups bears no nitrogen atoms which, based on an aqueous comparative scale at 25° C., have a pKa value of less than 20.

It is to be noted that the definitions selected above for reasons of simplicity also encompass the corresponding tertiary ammonium fluorides and N-heteroarylium fluorides and the corresponding polyfluorides, which occur in the reaction with hydrogen fluoride.

Preferred compounds of the formula (Ia) are those of the formula (I) as defined above and those of the formulae (Ib), (Ic), (Id) and (Ie)

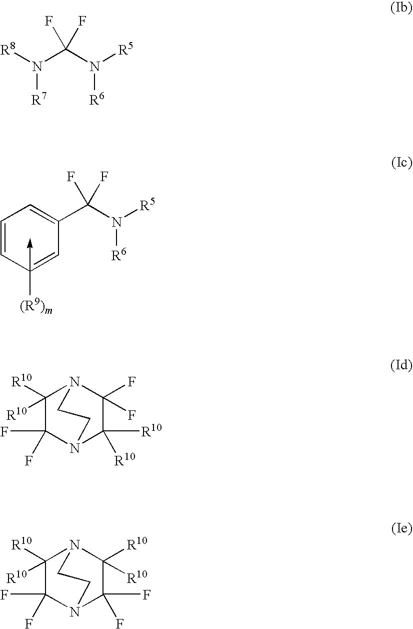

in which R⁵, R⁶, R⁷ and R⁸ are each as defined above, m is 0, 1, 2, 3 or 4 and R⁹ is a radical which is selected from the group of chlorine, fluorine, C₁-C₁₂-alkyl, C₁-C₁₂-fluoroalkyl, C₁-C₁₂-fluoroalkoxy, C₁-C₁₂-fluoroalkylthio, C₁-C₁₂-alkoxy and di(C₁-C₈-alkyl)amino and R¹⁰ is in each case independently hydrogen or C₁-C₁₂-alkyl.

As a compound of the formula (Ib), special mention should be made of 2,2′-difluoro-1,3-dimethylimidazolidine. As a compound of the formula (Ic), special mention should be made of N,N-diethyl-α,α-difluorophenylmethanamine, N,N-dimethyl-α,α-difluoro-phenylmethanamine, N,N-diisopropyl-α,α-difluorophenylmethanamine and diethyl-α,α-difluoro(4-chlorophenyl)methanamine. As a compound of the formula (Id), special mention should be made of [2.2.2]-2,2,5,5-tetrafluoro-3,3,6,6-tetramethyl-1,4-diazabicyclooctane. As a compound of the formula (Ie), mention should be made [2.2.2]-2,2,6,6-tetrafluoro-3,3,5,6-tetramethyl-1,4-diazabicyclooctane.

Preferred aprotic tertiary amines are those of the formulae (IVa) and (IVb)

NR¹¹R¹²R¹³ (IVa)

(R¹⁴)₂N-L-N(R¹⁴)₂ (IVb)

in which R¹¹, R¹² and R¹³ are each independently C₁-C₁₂-alkyl or [(C₂-C₁₂-alkylene)-O]ₙ(C₁-C₁₂-alkyl)] where n=1 to 5, or two or three of the R¹⁰, R¹¹ and/or R¹² radicals with the nitrogen atom form a mono- or bicyclic radical having a total of 3 to 12 or 5 to 15 carbon atoms respectively, L is C₂-C₆-alkylene and the R¹⁴ radicals are each independently C₁-C₈-alkyl or two radicals together are C₂-C₆-alkylene.

In formula (IVa), R¹¹, R¹² and R¹³ are preferably each independently C₁-C₁₂-alkyl, more preferably each identically C₁-C₈-alkyl.

Particularly preferred aprotic tertiary amines are triethylamine, tetramethylethylenediamine and [2.2.2]-1,4-diazabicyclooctane.

Preferred N-heterocyclic compounds are optionally substituted pyridine and quinoline, and particular preference is given to pyridine.

In the context of the invention, very particular preference is given to using triethylamine.

The molar ratio of aprotic tertiary amine or N-heteroaromatic compound to compounds of the formula (Ia) is, for example and with preference, 0.1:1 to 20:1, preferably 1:1 to 10:1 and more preferably 1:1 to 5:1.

The molar ratio of hydrogen fluoride to aprotic tertiary amine or N-heteroaromatic compounds is, for example and with preference, 0.2:1 to 10:1 per nitrogen atom.

The following is an illustrative but non-limiting description of the processes for preparing the mixtures and compounds of the invention. The inventive mixtures comprising compounds of the formula (Ia), at least one aprotic tertiary amine or N-heteroaromatic compound and hydrogen fluoride are obtainable, for example, by mixing the compounds of the formula (Ia) with aprotic tertiary amine or N-heteroaromatic compounds and hydrogen fluoride, or by mixing the compounds of the formula (Ia) with mixtures of aprotic tertiary amine or N-heteroaromatic compounds and hydrogen fluoride, which are also commercially obtainable in various compositions, for example (NEt₃.3 HF) or (pyridine 9HF).

The compounds of the formula (I) can be prepared in a particularly advantageous manner by converting compounds of the formula (V)

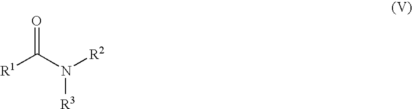

in which R¹, R² and R³ are each as defined above including the areas of preference specified, as follows:
in one step, a), using halogenating agents, to compounds of the formula (VI)

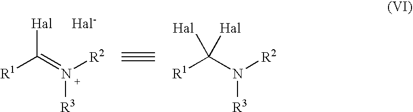

in which Hal is in each case independently chlorine or bromine and
in one step, b), converting the compounds of the formula (VI), using ionic fluoride, to compounds of the formula (I).

Preferred halogenating agents for step a) are phosphorus pentachloride, phosphorus pentabromide, thionyl chloride, thionyl bromide, phosgene and/or oxalyl chloride, and even greater preference is given to phosphorus pentachloride, thionyl chloride, phosgene and/or oxalyl chloride.

The molar ratio of halogenating agents to compounds of the formula (V) is, for example and with preference, 0.9:1 to 10:1, preferably 1:1 to 2:1 and more preferably 1.02:1 to 1.5:1.

The solvents used for step a) may be aliphatic, alicyclic or aromatic, optionally halogenated hydrocarbons, for example benzine, benzene, toluene, xylene, chlorobenzene, the isomeric dichlorobenzenes, petroleum ether, hexane, cyclohexane, dichloromethane, chloroform and/or ethers such as diethyl ether, diisopropyl ether, dioxane, tetrahydrofuran or ethylene glycol dimethyl or diethyl ether.

The reaction temperature in step a) may be, for example, −20° C. up to the boiling point of the solvent used at the reaction pressure, but has a maximum of 150° C., preferably −10° C. up to the boiling point of the solvent used at the reaction pressure, but a maximum of 50° C.

The reaction pressure in step a) may be, for example, 0.8 to 20 bar, preferably 0.9 to 3 bar, and even greater preference is given to ambient pressure.

The workup after the reaction may be effected, for example, by distilling off all volatile constituents and drying the residue under high vacuum.

The compounds of the formula (VI) which are obtainable in step a), as indispensable intermediates for the preparative process mentioned, are likewise encompassed by the invention.

Compounds of the formula (VI) include:
1,1-dichloromethyl-N,N-dimethylamine, 1,1-dichloromethyl-N,N-diethylamine, 1,1-di-chloromethyl-N,N-diisopropylamine, 1,1-dichloro-N,N-2-trimethyl-1-propanamine, 1,1-dichloro-N,N-2,2-tetramethyl-1-propanamine, N,N-diethyl-α,α-dichloro-2,2-dimethyl-1-propanamine, N-(1,1-dichloromethyl)morpholine, N,N-diethyl-α,α-dichloro-3-pyridyl-methanamine, N,N-diethyl-α,α-dichloro-2-pyridylmethanamine and 2,2-dichloro-1,3,3-trimethylpyrrolidine.

In step b), the compounds for the formula (VI) are reacted with ionic fluoride.

Ionic fluorides are, for example, quaternary ammonium or phosphonium fluorides, and also alkali metal fluorides or mixtures of the compounds mentioned.

Examples of ammonium or phosphonium fluorides are those of the formula (VII),

(cation⁺)(F⁻) (VII)

in which
(cation⁺) is a cation of the formula (VIII)

[pnic(C₁-C₁₂-alkyl)_{q}(C₆-C₁₅-arylalkyl)ᵣ(C₃-C₁₄-aryl)S({(C₂-C₈-alkylene)-O]ᵥ—(C₁-C₈-alkyl)}ₜ)]⁺ (VIII)

where
pnic is nitrogen or phosphorus and
in which (q+r+s+t)=4.

However, preference is given to using alkali metal fluorides or mixtures of alkali metal fluorides, and particular preference is given to sodium fluoride, potassium fluoride and caesium fluoride, and very particular preference to sodium fluoride.

The molar ratio of ionic fluoride to compound of the formula (VI) used may be, for example, 0.7 to 5, preferably 0.9 to 2 and more preferably 1.1 to 1.7. The upper limits of the amount of ionic fluoride which can be used result merely from economic considerations.

Preference is given to carrying out step b) in an organic solvent. Examples of suitable organic solvents are: nitriles such as acetonitrile, propionitrile, benzonitrile, benzyl nitrile or butyronitrile; amides such as N,N-dimethylformamide, N,N-dimethylacetamide, N-ethylformanilide, N-methylpyrrolidone and dimethylimidazolidinone, and also the amides of sulfoxides used as starting compounds for the preparations of the compounds of the formula (VI), such as dimethyl sulfoxide, sulphones such as tetramethylenesulphone, polyethers such as 1,4-dioxane, ethylene glycol dimethyl ether, ethylene glycol diethyl ether, diethylene glycol dimethyl ether, diethylene glycol diethyl ether, benzotrifluorides or mixtures of such organic solvents.

The water content of the solvent in the process according to the invention is preferably a maximum of 0.2% by weight, preferably a maximum of 0.05% by weight. Preference is given to attaining such a water content by incipient distillation or drying in a manner known per se. When alkali metal fluorides are used, particular preference is given to drying or incipiently distilling the solvents simultaneously in the presence of the alkali metal fluoride used.

The reaction temperature in step b) may be, for example, 60° C. up to the boiling point of the solvent used at reaction pressure, but has a maximum of 180° C., preferably 110° C. up to the boiling point of the solvent used at reaction pressure, but has a maximum of 150° C.

The reaction pressure may be, for example, 0.8 to 30 bar, preferably 1 to 2 bar.

Optionally, the reactivity of the ionic fluorides can be modified by additives. Suitable additives are, for example, phase transfer catalysts.

Suitable phase transfer catalyst are, for example, crown ethers, such as 18-crown-6,12-crown-4, dibenzo-18-crown-6 or dibenzo-18-crown-4, cryptands such as cryptands [2.2.2] or podands such as polyglycol ethers or those of the formula (IX)

(cation⁺)(anion⁻) (IX)

in which
(cation⁺) has the above definition and areas of preference and
(anion⁻) is the anion of an organic or inorganic acid.

In the manner described, the compounds of the formula (I), after workup which is, for example, performed as for compounds of the formula (VI), are obtained in high yields and purity.

For the preparation of compounds of the formula (Ia) and in particular for the preparation of the inventive mixtures, it has been found to be particularly useful to convert the compounds of the formula (VI)
in one step, b*),
   in the presence of hydrogen fluoride and optionally to react the reaction mixture obtained in this way with aprotic tertiary amine which contains no fluorine atoms in the α-position to the nitrogen and/or N-heteroaromatic compound.

The expression “in the presence of hydrogen fluoride” includes the possibility of using mixtures of hydrogen fluoride with aprotic tertiary amines which contain no fluorine atoms in the α-position to the nitrogen and/or N-heteroaromatic compounds, in which the hydrogen fluoride is present in a molar excess. Such mixtures are, for example, the abovementioned (NEt₃.3HF) and (pyridine.9HF) mixtures.

However, preference is given to carrying out step b*) in such a way that the inventive mixtures are prepared in such a way that compounds of the formula (VI) are reacted with sufficient hydrogen fluoride and the reaction mixture obtained in this way is reacted with sufficient aprotic, tertiary amine which contains no fluoride atoms in the α-position to the nitrogen and/or N-heteroaromatic compound that the mixing ratios specified above for the inventive mixtures are adhered to. The areas of preference specified apply in the same manner.

The inventive compounds of the formula (I) and also the inventive mixtures are suitable in particular for preparing fluorine compounds from the corresponding hydroxyl compounds, and also for preparing geminal difluoride compounds from the corresponding carbonyl compounds.

The invention therefore also encompasses a process for preparing fluorinated compounds, which is characterized in that compounds containing hydroxyl and/or carbonyl groups are reacted with compounds of the formula (I) and/or the inventive mixtures.

Preferred compounds containing hydroxyl and/or carbonyl groups are those which contain at least one aliphatic hydroxyl group and/or at least one ketone group and/or at least one aldehyde group and/or one carboxyl group.

Particularly preferred compounds containing hydroxyl and/or carbonyl groups are those which contain one aliphatic hydroxyl group or one ketone group or one aldehyde group or carboxyl group.

The fluorinated compounds which can be prepared in accordance with the invention are suitable in particular for preparing pharmaceuticals, agrochemicals and liquid crystals.

The inventive compounds and mixtures have the advantage that they can be prepared simply and are storage-stable, and enable the conversion of hydroxyl and carbonyl compounds to the corresponding fluoro and/or difluoro compounds in high yields. The inventive process for preparing the abovementioned compounds or mixtures start from readily available reactants and afford the products in high yields.

### EXAMPLES

### Example 1

### Preparation of 1,1-dichloro-N,N-2,2-tetramethyl-1-propanamine

27.8 g (210 mmol) of N,N-dimethylpivalamide and 250 ml of tert-butyl methyl ether are initially charged at 20° C. under a protective gas atmosphere in a 3-necked flask equipped with precision glass stirrer. 27.7 g (220 mmol) of oxalyl chloride are added dropwise to this reaction mixture, and a colourless solid is deposited during the addition. On completion of addition, the mixture is stirred until the end of gas evolution (approx. 2 h) and, to complete the reaction, the mixture is heated to 40° C. for 0.5 h. After all volatile constituents have been removed in high vacuum, 1,1-dichloro-N,N-2,2-tetramethyl-1-propanamine is obtained as a colourless, hydrolysis-sensitive solid.

Yield: 38.0 g (207 mmol; 96%)

¹H NMR (CDCl₃): 0.99 (s broad, 9H, t-Bu-H), 3.46 (s, 6H, N(CH₃)₂).

¹³C NMR (CDCl₃): 28.1 (CH₃, 3C, t-Bu-CH₃), 29.5 (quat. C, 1C, t-Bu-C), 44.8 (CH₃, 1C, NCH₃), 51.1 (CH₃, 1C, NCH₃), 186.6 (quat. C, 1C, C—Cl) ppm.

### Example 2

### Preparation of N,N-diethyl-α,α-dichloro-3-pyridylmethanamine

18.5 g (104 mmol) of N,N-diethylnicotinamide and 150 ml of tert-butyl methyl ether are initially charged at 20° C. under a protective gas atmosphere in a 3-necked flask equipped with a precision glass stirrer. 13.5 g (106 mmol) of oxalyl chloride are added dropwise to this reaction mixture, and a colourless solid is deposited during the addition. On completion of addition, the mixture is stirred to 20° C. for 1 h and, to complete the reaction, is heated to reflux for a further 4 h. After cooling to 20° C., the solvent is removed under water jet vacuum, and the remaining residue is washed with a little cold Et₂O. After drying in high vacuum, N,N-diethyl-α,α-dichloro-3-pyridylmethanamine is obtained as a slightly yellow solid (m.p.: 113-115° C.).

Yield: 22.9 g (98.8 mmol; 95%)

¹H NMR (CDCl₃): 1.16 (s, 6H, —CH₃), 3.90 (s, 4H, —CH₂), 7.21 (s, 1H, arom.-H), 8.37 (s, 2H, arom.-H), 8.91 (s, 1H, arom.-H) ppm

¹³C NMR (CDCl₃): 12.7 (CH₃, 2C, —CH₃), 55.6 (—CH₂, 2C, NCH₂—), 124.8 (—CH, 1C, arom.-C), 129.3 (—CH, 1C, arom.-C), 138.5 (—CH, 1C, arom.-C), 147.5 (—CH, 1C, arom.-C), 153.3 (-quat. C, 1C, arom.-C), 171.7 (quart. C, 1C, C—Cl) ppm.

In a similar manner to Example 1 and 2, the following were prepared: 1,1-dichloromethyl-N,N-dimethylamine (Example 3), 1,1-dichloromethyl-N,N-diethylamine (Example 4), 1,1-dichloromethyl-N,N-diisopropylamine (Example 5), 1,1-dichloro-N,N-2-trimethyl-1-propanamine (Example 6), N,N-diethyl-α,α-dichloro-2,2-dimethyl-1-propanamine (Example 7), N-(1,1-dichloromethyl)morpholine (Example 8), 1,1-dichloro-N,N-dimethyl(p-chlorophenyl)methanamine (Example 9), 1,1-dichloro-N,N-diisopropylphenyl-methanamine (Example 10), N,N-dimethyl-α,α-dichloro-2-pyridylmethanamine (Example 11) and 2,2-dichloro-1,3,3-trimethylpyrrolidine (Example 12).

The yield of Examples 3-12 are listed in Table 1:

TABLE 1

| Example | Formula | Yield |
|---|---|---|
| 3 | 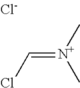 | 98% |
| 4 | 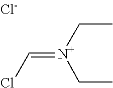 | 100% |
| 5 | 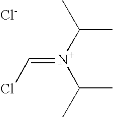 | 100% |
| 6 | 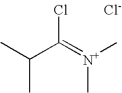 | 95% |
| 7 | 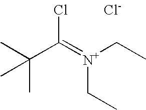 | 100% |
| 8 | 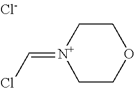 | 96% |
| 9 | 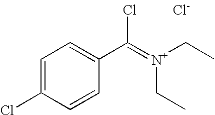 | 100% |
| 10 | 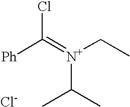 | 95% |
| 11 | 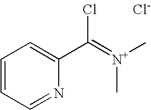 | 91% |
| 12 | 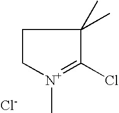 | 97% |

### Example 1a

### Preparation of 1,1-Difluoro-N,N-2,2-tetramethyl-1-propanamine 1a

17.8 g (424 mmol) of sodium fluoride are added under a protective gas atmosphere to a suspension of 19.5 g (107 mmol) of 1,1-dichloro-N,N-2,2-tetramethyl-1-propanamine from Example 1 in 75 ml of dimethylimidazolidinone and stirred at 20° C. for 25 h. The inorganic salts are filtered off under a protective gas atmosphere and washed twice with 20 ml of dimethylimidazolidinone each time. The crude product is condensed over under high vacuum from the reaction solution into a receiver cooled to −78° C. and, after subsequent fractional distillation, under reduced pressure (b.p.: 62° C./55 mbar), affords 1,1-difluoro-N,N-2,2-tetramethyl-1-propanamine as a slightly yellow liquid.

Yield: 13.6 g (90 mmol; 84%)

¹H NMR (CDCl₃): 1.00 (s broad, 9H, t-Bu-H), 2.26 (t, 6H, ⁴J_{HF}=1.95 Hz, N(CH₃)₂) ppm.

¹³C NMR(C₆D₆): 25.7 (s, CH₃, 3C, t-Bu-CH₃), 38.3 (t, CH₃, ³J_{CF}=6.03 Hz, N(CH₃)₂), 40.0 (t, quat. C, 1C, ²J_{CF}=29.8 Hz, t-Bu-C), 128.6 (t, CF₂, 1C, ¹J_{CF}=258.1 Hz) ppm.

¹⁹F NMR (CDCl₃): −97.5 (s, —CF₂) ppm.

In a similar manner, Examples 2a to 12a were carried out. The parameters and yields are reported in Table 2.

TABLE 2

| Ex- | | Time | Temp | | Yield | |
|---|---|---|---|---|---|---|
| ample | Compound | [h] | [° C.] | Solvent | [%] | b.p. |
| 1a | 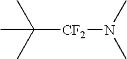 | 20 | 20 | CH₃CN | 84 | 62° C. 55 Torr |
| 2a | 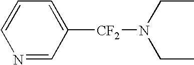 | 12 | 65-75 | CH₃CN | 74 | 55° C. 0.05 Torr |
| 3a | 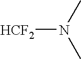 | 14-16 | 20 | DMF | 75 | 55° C. |
| 4a | 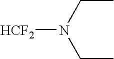 | 24 | 20 | Et₂NCHO | 63 | 41° C. 105 mm Hg |
| 5a | 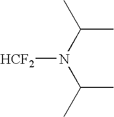 | 20 | 20 | (i-Pr)₂NCHO | 77 | 60° C. 65 Torr |
| 6a | 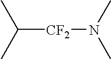 | 18-20 | 20 | CH₃CN | 61 | 42° C. 48 Torr |
| 7a | 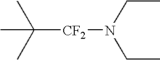 | 20 | 80 | CH₃CN | 71 | 64° C. 35 Torr |
| 8a | 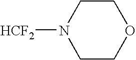 | 24 | 20 | DMI | 89 | — |
| 9a | 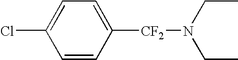 | 24 | 70 | CH₃CN | 71 | — |
| 10a | 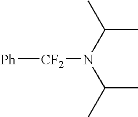 | 18 | 80 | CH₃CN | 79 | — |
| 11a | 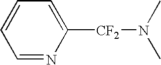 | 20 | 20 | DMI/Me₄N + F— | 95 | — |
| 12a | 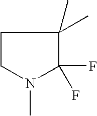 | 24 | 40 | DMI | 80.1 | 70° C. 25 Torr |

### Example 3b

### Preparation of Fluorinating Reagents Comprising 1,1-difluoromethyl-N,N-dimethyl-amine (3b)

A high-pressure vessel is initially charged with 10 g (64 mmol) of 1,1-dichloromethyl-N,N-dimethylamine under a protective gas atmosphere and cooled to 0° C. 5.6 ml (320 mmol) of HF are then metered in and the mixture is stirred for 3 h with cooling. On completion of reaction, the excess of HF and HCl which has been formed is removed under high vacuum. 8.9 ml (64 mmol) of triethylamine are added to the reaction mixture to obtain 17.8 g (64 mmol) of a mixture comprising Et₂N═CHF⁺HF₂⁻.HNEt₃⁺.HF₂⁻ (3b) as a slightly yellow liquid.

¹⁹F NMR (CD₂Cl₂): −89.2 (br s, 1F, CHF⁺), −167.7 (br s, 4F, HF₂⁻) ppm.

### Example 5b

### Preparation of Fluorinating Reagents Comprising 1,1-difluoromethyl-N,N-diisopropylamine (5b)

A polyethylene [flask] is initially charged under a protective gas atmosphere with 10.4 g (68.9 mmol) of 1,1-difluoromethyl-N,N-diisopropylamine and cooled to 0° C. 11.1 g (68.9 mmol) of NEt₃.3HF are then metered in within 2 min and the mixture is stirred for a further 20 min at this temperature. The initially liquid-crystal reaction mixture is allowed to cool to 20° C., is heated for homogenization at 40° C. for 0.5 h and is allowed to cool again to 20° C. This results in 21.5 g (68.9 mmol) of i-Prop₂N═CHF⁺.HF₂⁻HNEt₃⁺.HF₂⁻ (5b) having a melting point of 37-40° C.

¹⁹F NMR (CD₂Cl₂): −86.7 (br s, 1F, CHF⁺), −158.5 (br s, 4F, HF₂⁻) ppm.

### Reactions of Alcohols with α,α-difluoroamines

### Example 13 (for Comparison)

6.8 g (50 mmol) of 2,2-difluoro-1,3-dimethylimidazolidine are initially charged under a protective gas atmosphere and a solution of 5.5 g (45 mmol) of 1-phenylethanol in 20 ml of CH₃CN is added dropwise thereto. The reaction mixture is stirred at 20° C. for 6 h. After the end of the reaction, the mixture is admixed with 30 ml of 3% Na₂CO₃ solution and is extracted 3 times with 50 ml of n-pentane each time. After drying the combined organic phases over Na₂SO₄, the volatile constituents are removed. The residue is subsequently distilled and affords 2.9 g (23 mmol; 51%) of 1-fluoroethylbenzene (b.p.: 52° C./20 mbar).

¹H NMR (CDCl₃): 1.60 (dd, 3H, ³J_{HH}=6.5 Hz, ³J_{HF}=24.1 Hz, —CH₃), 5.57 (dq, 1H, J_{HH}=6.5 Hz, ²J_{HF}=47.8 Hz, —CHF), 7.18-7.43 (m, 5H, arom-H) ppm.

¹⁹F NMR (CDCl₃): −168.2 (dq, 1F, ²J_{HF}=47.8 Hz, ³J_{HF}=24.0 Hz, —CHF) ppm.

GC-MS: 124 [M⁺], 109 [M⁺-CH₃]

### Example 14

A solution of 9.51 g (63 mmol) of 1,1-difluoromethyl-N,N-diisopropylamine (5a) is initially charged under a protective gas atmosphere. A solution of 7.32 g (60 mmol) of 1-phenyethanol in 30 ml of CHCl₃ is added dropwise to this stirred solution, and the mixture is heated to 60° C. and stirred for 6 h. After the end of the reaction, the mixture is cooled to 20° C., 100 ml of ice-water are added and the aqueous phase is extracted twice with 50 ml of CHCl₃ each time. The combined organic phases are dried over Na₂SO₄, filtered off and concentrated. The residue is subsequently distilled and affords 6.45 g (52 mmol; 87%) of 1-fluoroethylbenzene (b.p.: 52° C./20 mbar).

### Reaction of Alcohols With Fluorinating Reagents Comprising α,α-difluoroamines

### Example 15

In a PE vessel, 0.83 g (6.8 mmol) of 1-phenylethanol are added dropwise within 5 min under a protective gas atmosphere to a solution of 2.32 g (7.56 mmol) of i-Prop₂N═CHF⁺HF₂⁻.HNEt₃⁺.HF₂⁻ (5b) in 10 ml of CH₂Cl₂. The mixture is stirred at 20° C. for several hours and the conversion is analyzed by ¹⁹F NMR (Reference: PhCF₃). After 2.5 h, 81% of 1-fluoroethylbenzene are obtained, and 96% of product are obtained after 24 h of stirring time.

### Example 16

A PE vessel is initially charged under a protective gas atmosphere with a solution of 12.4 g (44.4 mmol) of Et₂N═CHF⁺HF₂⁻.HNEt₃.HF₂⁻ (3b) and 9.88 g (93.2 mmol) of benzaldehyde are added dropwise thereto within 10 min. The mixture is stirred at 80° C. for several hours and the conversion is analysed by means of ¹⁹F NMR (Reference: PhCF₃). After 5 h of stirring time, 85% of product are obtained.

### Example 17

A solution of 7.05 g (33 mmol) of 1,1-difluoromethyl-N,N-diisopropylamine in 25 ml of CH₂Cl₂ is initially charged at −15° C. under a protective gas atmosphere. A solution of 10.0 g (31 mmol) of N-tert-butoxycarbonyl-trans-4-hydroxy-L-proline benzyl ester in 25 ml of CH₂Cl₂ is added dropwise to the stirred solution, and the mixture is allowed to come to room temperature and is heated to reflux with stirring for 3.5 h. After the end of the reaction, the mixture is cooled to 20° C., semi-saturated NaHCO₃ solution is added and the aqueous phase is extracted twice with 50 ml of CH₂Cl₂ each time. The combined organic phases are dried over Na₂SO₄, filtered off and concentrated. The residue is subsequently distilled and affords 6.15 g (19 mmol; 61%) of N-tert-butoxycarbonyl-trans-4-fluor-L-proline benzyl ester.

Reactions with α,α-difluoroamines

Further reactions of alcohols with 1,1-difluoro-N,N,2,2-tetramethyl-1-propanamine (1a) are reported in Table 3.

TABLE 3

| | | Temp. | Time | | | Yield |
|---|---|---|---|---|---|---|
| | Substrate | [° C.] | [h] | Solvent | Product | [%] |
| 18 | 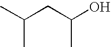 | 0 | 72 | CH₂Cl₂ | 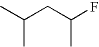 | 57 |
| 19 | 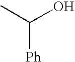 | 60 | 2 | CHCl₃ | 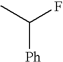 | 75 |
| 20 | 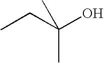 | 0 | 3 | CH₂Cl₂ | 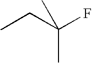 | 45 |
| 21 | n-C₇H₁₅—OH | 60 | 2 | CHCl₃ | n-C₇H₁₅—F | 57 |
| 22 | 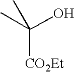 | 60 | 6 | CHCl₃ | 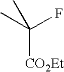 | 61 |
| 23 | 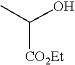 | 100 | 0.2 | Toluene | 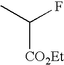 | 81 |

Reactions of alcohols and aldehydes with 1,1-difluoromethyl-N,N-diethylamine (4a) are reported in Table 4.

TABLE 4

| | | Temp. | Time | | | Yield |
|---|---|---|---|---|---|---|
| | Substrate | [° C.] | [h] | Solvent | Product | [%] |
| 23 | 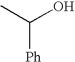 | 20 | 14 | CH₂Cl₂ | 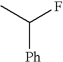 | 75 |
| 24 | 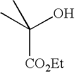 | 60 | 6 | CHCl₃ | 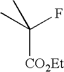 | 67 |
| 25 | 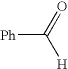 | 85 | 4 | — | 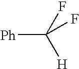 | 35* |

*Reaction with 2 eq. of α,α-difluoroamine

Reactions of alcohols with 1,1-difluoromethyl-N,N-diisopropylamine (5a) are reported in Table 5.

TABLE 5

| | | Temp. | Time | | | Yield |
|---|---|---|---|---|---|---|
| | Substrate | [° C.] | [h] | Solvent | Product | [%] |
| 26 | 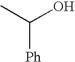 | 60 | 1 | CHCl₃ | 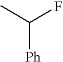 | 87 |
| 27 | 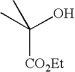 | 20 | 24 | CHCl₃ | 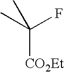 | 51 |
| 28 | 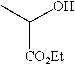 | 100 | 0.2 | Toluene | 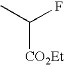 | 81 |

Reactions of alcohols with 1,1-difluoro-N,N-dimethylphenylmethanamine (10a) (as a comparison) are reported in Table 6.

TABLE 6

| | | Temp. | Time | | | Yield |
|---|---|---|---|---|---|---|
| | Substrate | [° C.] | [h] | Solvent | Product | [%] |
| 29 | n-C₇H₁₅—OH | 20 | 12 | CH₃CN₃ | n-C₇H₁₅—F | 18 |
| 30 | 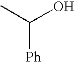 | 20 | 3 | CHCl₃ | 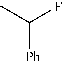 | 51 |
| 31 | 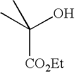 | 60 | 1.5 | CHCl₃ | 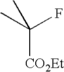 | 45 |
| 32 | 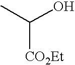 | 75 | 1.5 | CHCl₃ | 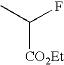 | 40 |
| 33 | 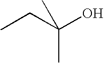 | 0 | 24 | CHCl₃ | 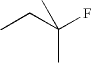 | 25 |
| 34 | 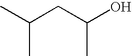 | 0 | 72 | CHCl₃ | 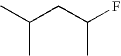 | 30 |

*addition of the α,α-difluoroamine

Reactions of alcohols with N,N-diethyl-β,α-difluoro-3-pyridinemethanamine (2a) are reported in Table 7:

TABLE 7

| | | Temp. | Time | | | Yield |
|---|---|---|---|---|---|---|
| | Substrate | [° C.] | [h] | Solvent | Product | [%] |
| 35 | 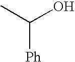 | 20 | 12 | CH₂Cl₂ | 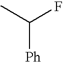 | 73 |
| 36 | 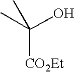 | 40 | 16 | CHCl₃ | 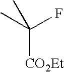 | 81 |
| 37 | 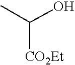 | 60 | 2 | CHCl₃ | 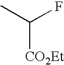 | 60 |

Reactions of alcohols with N,N-dimethyl-α,α-difluoro-2-pyridinemethanamine (11a) are reported in Table 8:

TABLE 8

| | | Temp. | Time | | | Yield |
|---|---|---|---|---|---|---|
| | Substrate | [° C.] | [h] | Solvent | Product | [%] |
| 38 | 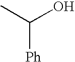 | 20 | 12 | CH₂Cl₂ | 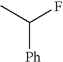 | 60 |
| 39 | 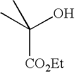 | 20 | 12 | CH₂Cl₂ | 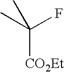 | 71 |
| 40 | 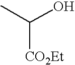 | 60 | 2 | CHCl₃ | 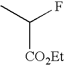 | 65 |

Reactions of alcohols with 2,2-difluoro-1,3,3-trimethylpyrrolidine (12a) are reported in Table 9:

TABLE 9

| | | Temp. | Time | | | Yield |
|---|---|---|---|---|---|---|
| | Substrate | [° C.] | [h] | Solvent | Product | [%] |
| 41 | 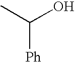 | 20 | 12 | CHCl₃ | 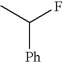 | 80 |

Reactions with Fluorinating Reagents Comprising α,α-difluoroamine

Reactions of alcohols with i-Prop₂N═CHF⁺HF₂⁻.HNEt₃⁺.HF₂⁻ (5b) are reported in Table 10:

TABLE 10

| | | Temp. | Time | | | Yield |
|---|---|---|---|---|---|---|
| | Substrate | [° C.] | [h] | Solvent | Product | [%] |
| 42 | 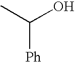 | 20 | 24 | CH₂Cl₂ | 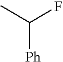 | 96* |

Reactions of aldehydes with 2eq of Et₂N═CHF⁺HF₂⁻.HNEt₃.HF₂⁻ (3b) are reported in Table 11:

TABLE 11

| | | Temp. | Time | | | Yield |
|---|---|---|---|---|---|---|
| | Substrate | [° C.] | [h] | Solvent | Product | [%] |
| 43 | 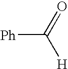 | 80-85 | 4-5 | — | 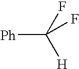 | 85* |

*by ¹⁹F NMR

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Mixtures comprising
compounds of the formula (Ia) 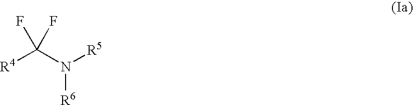
in which
R⁴ is hydrogen, C₁-C₁₂-alkyl, [(C₂-C₁₂-alkylene)-O]ₙ(C₁-C₁₂-alkyl)] where n=1 to 5, C₃-C₁₄-aryl or NR⁷R⁸ where R⁷ and R⁸ are each independently C₁-C₈-alkyl, or NR⁷R⁸ as a whole is a 4- to 7-membered cyclic radical having a total of 3 to 12 carbon atoms and
R⁵ and R⁶ are each independently C₁-C₁₂-alkyl or are together part of a cyclic radical having a total of 4 to 12 carbon atoms or
R⁴ and R⁵ and/or R⁶ together are part of a cyclic radical having a total of 4 to 12 carbon atoms,
at least one aprotic, tertiary amine which contains no fluorine atoms in the α-position to the nitrogen and/or at least one N-heteroaromatic compound and
hydrogen fluoride.

2. Mixtures according to claim 1, characterized in that the molar ratio of aprotic tertiary amine and/or N-heteroaromatic compound to compounds of the formula (Ia) is 0.1:1 to 20:1.

3. Mixtures according to claim 1, characterized in that the molar ratio of hydrogen fluoride to aprotic tertiary amine and/or N-heteroaromatic compound is 0.2:1 to 10:1 per nitrogen atom.

4. Mixtures according to claim 1, characterized in that the compounds of formula (Ia) are those of the formula (I) as defined in claim 1 or those of the formulae (Ib), (ic), (Id) or (Ie) 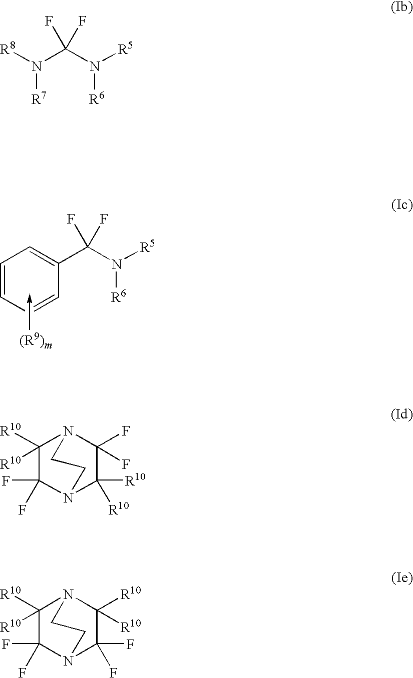
in which
R⁵, R⁶, R⁷ and R⁸ are each as defined in claim 6,
m is 0, 1, 2, 3 or 4 and
R⁹ is a radical which is selected from the group of chlorine, fluorine, C₁-C₁₂-alkyl, C₁-C₁₂-fluoroalkyl, C₁-C₁₂-fluoroalkoxy, C₁-C₁₂-fluoroalkylthio, C₁-C₁₂-alkoxy and di(C₁-C₈-alkyl)amino and
R¹⁰ is in each case independently hydrogen or C₁-C₁₂-alkyl.

5. Process for preparing compounds of the formula (I) 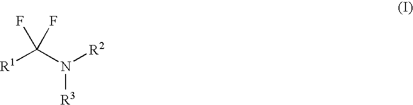
in which
R¹ is C₂-C₁₂-alkyl,
R² and R³ are each independently C₁-C₁₂-alkyl, comprising
in step a), converting with halogenating agents the compounds of the Formula (V) 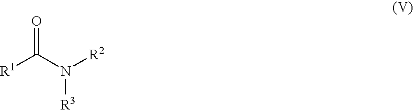
in which
R¹, R² and R³ are each as defined in claim 1
to compounds of the formula (VI) 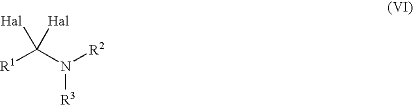
in which
Hal is in each case independently chlorine or bromine and
in step, b), converting the compounds of the formula (VI), using ionic fluoride, to compounds of the formula (I).

6. Process according to claim 5, characterized in that the halogenating agents used for step a) are phosphorus pentachloride, phosphorus pentabromide, thionyl chloride, thionyl bromide, phosgene and/or oxalyl chloride.

7. Process according to claim 5, characterized in that the ionic fluorides used are quaternary ammonium or phosphonium fluorides or else alkali metal fluorides or mixtures of the compounds mentioned.

8. Process according to claim 5, characterized in that the reactivity of the ionic fluorides is modified by additives.

9. Process for preparing mixtures according to claim 1, comprising converting compounds of the formula (VI) 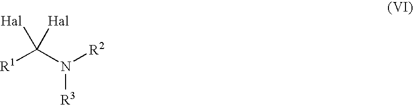
in which
Hal is in each case independently chlorine or bromine in the presence of hydrogen fluoride and optionally reacting the resulting reaction mixture with aprotic tertiary amine which contains no fluorine atoms in the α-position to the nitrogen and/or N-heteroaromatic compound.

10. Process according to claim 9, characterized in that compounds of the formula (VI) are reacted with sufficient hydrogen fluoride and sufficient aprotic, tertiary amine which contains no fluorine atoms in the α-position to the nitrogen and/or N-heteroaromatic compound is added to the resulting reaction mixture to provide the molar ratio of aprotic tertiary amine and/or N-heteroaromatic compound to compounds of the formula (Ia) is 0.1:1 to 20:1, and the molar ratio of hydrogen fluoride to aprotic tertiary amine and/or N-heteroaromatic compound is 0.2:1 to 10:1 per nitrogen atom.

11. Compounds of formula (VI) selected from the group consisting of 1,1-dichloromethyl-N,N-dimethylamine, 1,1-dichloromethyl-N,N-diethylamine, 1,1-dichloromethyl-N,N-diisopropylamine, 1,1-dichloro-N,N-2-trimethyl-1-propanamine, 1,1-dichloro-N,N-2,2-tetramethyl-1-propanamine, N,N-diethyl-α,α-di-chloro-2,2-dimethyl-1-propanamine, N-(1,1-dichloromethyl)morpholine, 1,1-di-chloro-N,N-dimethylphenylmethanamine, N,N-diethyl-α,α-dichloro-3-pyridyl-methanamine, N,N-diethyl-α,α-dichloro-2-pyridylmethanamine and 2,2-dichloro-1,3,3-trimethylpyrrolidine.

12. Process for preparing fluorinated compounds, characterized in that compounds containing hydroxyl and/or carbonyl groups are reacted with compounds according to claim 1.

13. Process for preparing fluorinated compounds, characterized in that compounds containing hydroxyl and/or carbonyl groups are reacted with mixtures according to claim 1.

14. Process according to claim 12, characterized in that the compounds containing hydroxyl and/or carbonyl groups are those which contain at least one aliphatic hydroxyl group and/or at least one ketone group and/or at least one aldehyde group and/or one carboxyl group.

15. A process for preparing fluorine compounds from the corresponding hydroxyl compounds or for preparing geminal difluoro compounds from the corresponding carbonyl compounds comprising providing compounds according to claim 1.

16. Process for preparing fluorine compounds from the corresponding hydroxyl compounds or for preparing geminal difluoro compounds from the corresponding carbonyl compounds comprising providing mixtures according to claim 1.

17. A process for preparing pharmaceuticals, agrochemicals or liquid crystals comprising providing fluorinated compounds which have been prepared according to claim 12.
